(19) United States
(12) Patent Application Publication
(10) Pub. No.: US 2025/0182934 A1
(43) Pub. Date: Jun. 5, 2025

(54) METHODS OF MAKING TISSUE STIMULATION APPARATUS

(71) Applicant: The Alfred E. Mann Foundation for Scientific Research, Valencia, CA (US)

(72) Inventors: Christopher Reed Jenney, Valencia, CA (US); Neil Talbot, La Crescenta, CA (US); Joseph L. Calderon, Santa Clarita, CA (US); Sahar Elyahoodayan, Los Angeles, CA (US); William Andrew Brandt, Castaic, CA (US)

(73) Assignee: The Alfred E. Mann Foundation for Scientific Research, Valencia, CA (US)

(21) Appl. No.: 18/939,479

(22) Filed: Nov. 6, 2024

Related U.S. Application Data
(60) Provisional application No. 63/606,415, filed on Dec. 5, 2023.

(51) Int. Cl. H01B 13/00; A61N 1/05; B08B 7/00; B24C 1/08

(52) U.S. Cl. CPC H01B 13/0036; A61N 1/0556; B08B 7/00; B24C 1/08

(57) ABSTRACT

A method of forming a nerve cuff by combining a plurality of electrically conductive members with respective rear surfaces and MgSulfate blasted front surfaces with a nerve cuff body, which includes a respective plurality of windows, in such a manner that exposed portions of the MgSulfate blasted front surfaces are within the windows.

### CROSS-REFERENCE TO RELATED APPLICATION

This application claims the benefit of U.S. Provisional Application No. 63/606,415, filed Dec. 5, 2023, and entitled “Methods of Making Tissue Stimulation Apparatus,” which is incorporated herein by reference.

### BACKGROUND OF THE INVENTIONS

### 1. Field of Inventions

The present inventions relate generally to tissue stimulation apparatus and method of making the same.

### 2. Description of the Related Art

There are many instances where tissue is stimulated to produce a desired therapeutic result. Although the present inventions are not limited to particular stimulation apparatus, one exemplary type of stimulation apparatus is an electrode lead that is used to stimulate the nerve fascicles of the hypoglossal nerve (HGN) to treat obstructive sleep apnea (OSA) and the present inventions are described in this exemplary context. OSA is a highly prevalent sleep disorder that is caused by the collapse of or increase in the resistance of the pharyngeal airway, often resulting from tongue obstruction.

Some proposed methods of alleviating apneic events involve the use of neurostimulators to open the upper airway. Such therapy involves stimulating the nerve fascicles of the hypoglossal nerve (HGN) that innervate the intrinsic and extrinsic muscles of the tongue in a manner that prevents retraction of the tongue, which would otherwise close the upper airway during the inspiration portion of the respiratory cycle. In some instances, the trunk of the HGN is stimulated with a nerve cuff, including a cuff body and a plurality of electrically conductive contacts (sometimes referred to as “electrodes”) on the cuff body, that is positioned around the HGN trunk. To that end, some nerve cuffs are pre-shaped to a furled state, may assume slightly less furled states, and may be unfurled to a flattened state. The HGN trunk nerve cuff may be configured in such a manner that it can be used to selectively stimulate nerve fascicles which innervate muscles that extend the tongue, while avoiding other nerve fascicles, with what is predominantly radial vector stimulation. Stimulation energy is supplied by way of cables that are connected to the electrically conductive contacts. HGN branches may also be stimulated. For example, an HGN GM branch may be stimulated with what is predominantly axial vector stimulation.

The contacts of at least some nerve cuffs are defined by electrically conductive members that are located between two non-conductive layers, i.e., a relatively thin front layer and a relatively thick rear layer, with the front layer including openings (or “windows”) that expose portions of the conductive members. The portions of window-containing layer that cover the remainders of the conductive members, and that border the windows, are referred to herein as “window frames.” The conductive members are connected by cables to stimulation circuitry. Exemplary nerve cuffs are illustrated and described in U.S. Pat. Pub. Nos. 2018/0318577A1, 2018/0318578A1, 2019/0060646A1,2019/0282805A1 and 2022/0313987A1, which are incorporated herein by reference in their entirety.

### SUMMARY

The present inventors have determined that stimulation apparatus, such as nerve cuffs, are susceptible to improvement. For example, the present inventors have determined that certain electrically conductive materials with otherwise desirable properties (e.g., platinum-iridium) do not bond well with non-conductive materials that have desirable mechanical properties (e.g., silicone) or, when employed, the adhesive (e.g., silicone adhesive) that is used to bond non-conductive layers that are formed from materials that have desirable mechanical properties. The less than optimal bond, coupled with the thinness of the window frames and the stress that is applied to the conductive members when the nerve cuff is in manipulated (e.g., furled, unfurled, or twisted), may cause delamination of the nerve cuff at the window frames, exposure of internal components not intended for direct bodily fluid contact and, in some instances, dislodgement of the conductive members. Exposure of the internal components to bodily fluid may result in, for example, electrochemically driven oxidation of the cables during stimulation pulses and an increased risk of galvanic corrosion between the conductive member and cable materials in the crimp joints connecting the cables to the conductive members. In addition, exposure of the normally completely covered rear side of the conductive members to bodily fluid reduces the effective charge density which can result in less effective stimulation. Accordingly, the present inventors have determined that it would be desirable to provide nerve cuffs that, among other things, reduce the likelihood of delamination at the window frames.

Other issues identified by the present inventors are associated with the effective surface area of conventional flat electrodes, i.e., the surface area value that takes into account the surface roughness of the electrodes as well as the geometric boundaries defined by the windows. In particular, the present inventors have determined that it would be desirable to increase the effective surface area of flat electrodes without increasing window size in order to increase the amount of stimulation energy that can be safely delivered and to also reduce electrode-electrolyte impedance, thereby reducing power consumption by the stimulation circuitry.

A method in accordance with at least one of the present inventions includes forming a nerve cuff by combining a plurality of electrically conductive members with respective rear surfaces and magnesium sulfate (“MgSulfate”) blasted front surfaces with a nerve cuff body, which includes a respective plurality of windows, in such a manner that exposed portions of the MgSulfate blasted front surfaces are within the windows.

A method in accordance with at least one of the present inventions includes MgSulfate blasting conductive material using first MgSulfate blasting parameters to form a MgSulfate blasted conductive surface, assembling a nerve cuff that includes a cuff body having a plurality of windows and the MgSulfate blasted conductive material positioned such that portions of the MgSulfate blasted conductive material are located within the windows, and removing contaminants from the conductive material located within the windows of the assembled nerve cuff by MgSulfate blasting the conductive material located within the windows using second MgSulfate blasting parameters that may be different than the first MgSulfate blasting parameters.

A method in accordance with at least one of the present inventions includes MgSulfate blasting a portion of a nerve cuff blank, that includes a cuff body and at least one coil-shaped conductive member embedded in the cuff body, in such a manner that a portion of the coil-shaped conductive member is exposed.

### BRIEF DESCRIPTION OF THE DRAWINGS

Detailed descriptions of exemplary embodiments will be made with reference to the accompanying drawings.

FIG. 1 is a plan view of a stimulation system in accordance with one embodiment of a present invention.

FIG. 2 is a plan view of a portion of the stimulation system illustrated in FIG. 1.

FIG. 3 is a cut-away anatomical drawing of the head and neck area illustrating the muscles that control movement of the tongue, the HGN and its branches that innervate these muscles, and the nerve cuff illustrated in FIG. 1 on the HGN trunk.

FIG. 4 is a plan view showing the nerve cuff illustrated in FIG. 1 on the HGN GM branch.

FIG. 5 is a front view of the nerve cuff illustrated in FIG. 1 in an unfurled state.

FIG. 6 is a rear, cutaway view of the nerve cuff illustrated in FIG. 1 in an unfurled state.

FIG. 7 is a section view of the nerve cuff illustrated in FIG. 1 in a furled state around a HGN branch.

FIG. 8 is a section of a magnesium sulfate (“MgSulfate”)-blasted tubular workpiece, a crimp tube and a lead wire in accordance with one embodiment of a present invention.

FIG. 9 is a section view of a MgSulfate blasted conductive member in accordance with one embodiment of a present invention.

FIG. 10 is an end view of a tubular workpiece in accordance with one embodiment of a present invention prior to undergoing a pre-assembly MgSulfate blasting process.

FIG. 11 is a top view of the tubular workpiece illustrated in FIG. 10 prior to undergoing a pre-assembly MgSulfate blasting process.

FIG. 12 is a magnified side view of the outer surface of the tubular workpiece illustrated in FIG. 10 prior to undergoing a pre-assembly MgSulfate blasting process.

FIG. 13 is a magnified side view of the outer surface of the tubular workpiece illustrated in FIG. 10 undergoing a pre-assembly MgSulfate blasting process.

FIG. 14 is a top view of the tubular workpiece illustrated in FIG. 10 after undergoing a pre-assembly MgSulfate blasting process.

FIG. 15 is a magnified side view of the outer surface of the tubular workpiece illustrated in FIG. 10 after undergoing a pre-assembly MgSulfate blasting process.

FIG. 16 is a section view of a portion of the nerve cuff illustrated in FIG. 1 prior to undergoing a post-assembly MgSulfate blasting process.

FIG. 17 is a magnified side view of the outer surface of a conductive member of the nerve cuff illustrated in FIG. 1 prior to undergoing a post-assembly MgSulfate blasting process.

FIG. 18 is a magnified side view of the outer surface of a conductive member of the nerve cuff illustrated in FIG. 1 undergoing a post-assembly MgSulfate blasting process.

FIG. 19 is a section view of a portion of the nerve cuff illustrated in FIG. 1 after undergoing a post-assembly MgSulfate blasting process.

FIG. 20 is a front view of the nerve cuff illustrated in FIG. 1 in an unfurled state.

FIG. 21 is a front view of a nerve cuff in accordance with one embodiment of a present invention in an unfurled state.

FIG. 22 is a side view of a portion of the nerve cuff illustrated in FIG. 21.

FIG. 23 is a perspective view of a portion of the nerve cuff illustrated in FIG. 21.

FIG. 24 is a rear, cutaway view of the nerve cuff illustrated in FIG. 21 in an unfurled state.

FIG. 25 is a front view of a nerve cuff blank in accordance with one embodiment of a present invention.

FIG. 26 is a section view take along line 26-26 in FIG. 25.

FIG. 27 is a section view showing a method involving the nerve cuff blank illustrated in FIG. 25 in accordance with one embodiment of a present invention. FIG. 28 is a section view of the result of the method illustrated in FIG. 27.

FIG. 29 is a front view of a nerve cuff in accordance with one embodiment of a present invention in an unfurled state.

FIG. 30 is a section view take along line 30-30 in FIG. 29.

FIG. 31 is an enlarge view of a portion of the nerve cuff illustrated in FIG. 29.

FIG. 32 is a section view of a nerve cuff blank in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

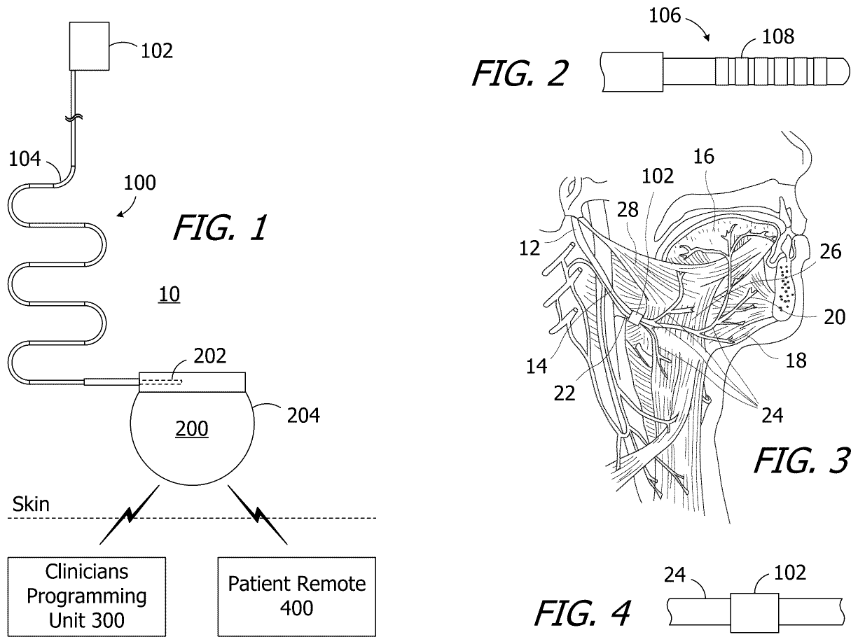

Referring to FIGS. 1 and 2, a stimulation system 10 in accordance with one embodiment of a present invention includes an electrode lead 100 and an implantable stimulator such as the implantable pulse generator (“IPG”) 200. A clinician's programming unit 300, a patient remote 400 and/or an IPG charger (not shown) may also be provided in some instances. Suitable IPGs, clinician's programming units and patient remotes are described, for example, in U.S. Pat. Pub. No. 2022/0313987A1. The exemplary electrode lead 100 includes a nerve cuff 102 and a lead body 104 that couples the nerve cuff 102 to the IPG 200 by way of lead connector 106, with a plurality contacts 108, on the proximal end of the lead body 104 and a corresponding connector receptacle 202 on the IPG 200. The nerve cuff 102 is configured in such a manner that it may be circumferentially disposed around either the HGN trunk or a HGN branch (e.g., the HGN GM branch) as is discussed below with reference to FIGS. 3 and 4. The lead body 104 may include one or more S-shaped sections in order to provide strain relief (as shown) or may be straight. The S-shaped sections accommodate body movement at the location within the neck where the lead body 104 is implanted, thereby reducing the likelihood that the HGN will be damaged due to unavoidable pulling of the electrode lead 100 that may result from neck movements. The accommodation provided by the S-shaped sections also reduces the likelihood of fatigue damage. Additionally, although the exemplary system 10 includes a single electrode lead 100, other embodiments may include a pair of electrode leads 100 for bilateral HGN stimulation and an IPG (not shown) with two connector receptacles.

Turning to FIG. 3, and as alluded to above, the nerve cuff 102 may be positioned around the trunk 14 of the HGN 12 and used to stimulate the muscles that anteriorly move the tongue 16 and, in particular, the fascicles of the HGN 12 that innervate the tongue protrusor muscles, such as the genioglossus 18 and/or the geniohyoid muscles 20. The nerve cuff 102 is positioned on the HGN trunk 14 at a position 22 proximal to the HGN branches 24. Although there are advantages to implanting the nerve cuff 102 at this proximal position 22, i.e., reduced surgical time and effort as well as reduced risk and trauma to the patient, it introduces the problem of inadvertently stimulating other fascicles of the HGN trunk 14 that innervate muscles in opposition to the genioglossus 18 and/or the geniohyoid muscles 20, i.e., the tongue retractor muscles, e.g., the hyoglossus 26 and styloglossus muscles 28, as well as the intrinsic muscles of the tongue 16. Accordingly, while some clinicians may desire to stimulate the HGN 12 at the HGN trunk 14, others may desire to stimulate the HGN at the GM branch 24. As illustrated in FIG. 4, the same nerve cuff 102 is configured in such a manner that it may be positioned the HGN GM branch 24 instead of the trunk 14.

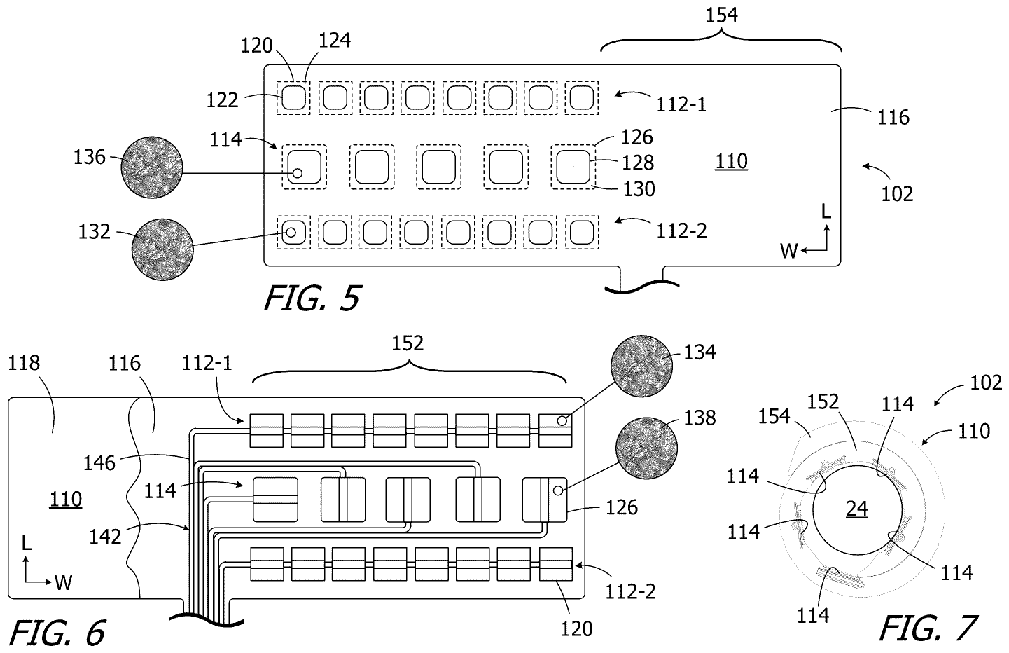

The exemplary nerve cuff 102 is shown in a flattened, unfurled state in FIGS. 5 and 6 and is shown in a furled (or “curled”) state in FIGS. 7. In the illustrated implementation, the nerve cuff 102 is pre-set (or “pre-shaped”) to the furled state and an external force may be used to partially or completely unfurl the nerve cuff 102. The nerve cuff 102 will return to the pre-shaped furled state when the force is removed and, as discussed below, may assume a variety furled states depending on the size of the HGN trunk or HGN branch that the nerve cuff 102 is placed around. Various examples of nerve cuffs that are capable of assuming different sizes are disclosed in aforementioned U.S. Pat. Pub. No. 2019/0060646A1.

The exemplary nerve cuff 102 illustrated in FIGS. 5-7 includes a cuff body 110 that defines a length L and a width W that is greater than the length, first and second pluralities of electrically conductive contacts (or “contacts”) 112-1 and 112-2 on the cuff body 110, and a plurality of electrically conductive contacts (or “contacts”) 114. The contacts 112-1 are spaced from one another in the length direction, as are the contacts 112-2. Contacts 112-1, 112-2 and 114 may also be referred to as “electrodes.” The contacts 112-1 are connected to one another in series and function as a single relatively wide contact. The contacts 112-2 are also connected to one another in series and function as a single relatively wide contact. The contacts 114 are not connected to one another in series and, as compared to the each of the pluralities of contacts 112-1 and 112-2, the contacts 114 are relatively narrow. Although the number may increase or decrease in the context of other nerve applications, at least five contacts 114 may be spaced from one another in the width direction and located between the electrically conductive contacts 112-1 and 112-2 (collectively “contacts 112”), and there are five relatively narrow contacts 114 in the illustrated embodiment. As used herein, “relatively wide” structures are structures that are longer in the width direction than structures that are referred to as “relatively narrow” and “relatively narrow” structures are structures that are shorter in the width direction than structures that are referred to as “relatively wide.”

In the implementation illustrated in FIGS. 5-7, the contacts 114 are centered relative to the contacts 112-1 and 112-2 and are aligned with one another in the length direction. In other implementations, the contacts may be non-centered relative to the relatively wide contacts 112-1 and 112-2 and/or offset from one another in the length direction. With respect to shape, and although the present inventions are not so limited, the individual contacts 112 are in the shape of rectangles with rounded corners, while the contacts 114 are squares with rounded corners. Other exemplary shapes are discussed below.

The cuff body 110 in the exemplary implementation may include a front layer 116 that will face the HGN trunk or branch and a rear layer 118 that will face away from the HGN trunk or branch. A plurality of conductive members 120, which form the contacts 112, are located between the front layer 116 and rear layer 118. The plurality of conductive members 120 are exposed by way of a plurality of windows 122 in the cuff body front layer 116. As discussed in greater detail below, the windows 122 are located inwardly of the outer perimeter (and outer edges) of the conductive members 120, which are shown in dashed lines in FIG. 5, and the exposed portions of the conductive members define the contacts 112. The portions of the front layer 116 that are located between the windows 122 and the outer perimeters (and outer edges) of the conductive members 120 define window frames 124 that hold the conductive members 120 against the rear layer 118 and between front and rear layers 116 and 118. Similarly, a plurality of conductive members 126, which form the contacts 114, are located between the front layer 116 and rear layer 118. The plurality of conductive members 126 are exposed by way of a plurality of windows 128 in the cuff body front layer 116. The windows 128 are located inwardly of the outer perimeter of the conductive members 126, which are shown in dashed lines in FIG. 5, and the exposed portions of the conductive members define the contacts 114. The portions of the front layer 116 that are located between the windows 128 and the outer perimeters (and outer edges) of the conductive members 126 define window frames 130 that hold the conductive members 126 against the rear layer 118 and between the front and rear layers 116 and 118.

The conductive members 120 each have a front surface 132 and a rear surface 134, while the conductive members 126 each have a front surface 136 and a rear surface 138. The front surfaces 132 and 136 face the nerve and have portions that are covered by the window frames 124 and 130 as well as portions that are exposed by the windows 122 and 128, while the rear surfaces 134 and 138 are covered by the rear layer 118. The front surfaces 132 and 136 have an effective surface area (“ESA”) that is at least twice the smooth surface area (“SSA”) that are defined by the outer perimeters of the associated conductive members 120 and 126, i.e., the areas of perfectly smooth surfaces having the size and shape of the front and rear surfaces. In at least some instances, including the illustrated embodiment, the rear surfaces 134 and 138 also have an ESA that is at least twice the SSA of the associated conductive members 120 and 126. Put another way, the ESA to SSA ratio is greater than that of conventional conductive members and is at least 1.5 in the exemplary implementations. For example, the ESA to SSA ratio of the front and rear surfaces in other implementations may be at least 1.5, or at least 2.0, or at least 3.0, or at least 4.0, or at least 5.0, or at least 6.0, or at least 7.0, or at least 8.0, or at least 9.0, or at least 10.0. Additionally, the portions of the front surfaces 132 and 136 that are exposed by the windows 122 and 128 have an effective surface area (“EPESA”) that is at least twice the area defined by the perimeters of the windows (“AW”). Put another way the EPESA to AW ratio is greater than that of conventional conductive member and window combinations and is at least 1.5 in the exemplary implementations. For example, the EPESA to AW ratio in other implementations may be at least 1.5, or at least 2.0, or at least 3.0, or at least 4.0, or at least 5.0, or at least 6.0, or at least 7.0, or at least 8.0, or at least 9.0, or at least 10.0.

Although the effective surface area of a surface is often correlated to the average roughness (Ra or Sa) of that surface, this correlation is not simple or linear in nature. The average roughness of the front surfaces 132 and 136 of conductive members 120 and 126 may be within a range of about 200 nm to about 2000 nm in some implementations. As used herein in the context of dimensions, the word “about” means±10-20%. For purposes of comparison, platinum-iridium foil may have a surface roughness of about 5-30 nm prior to a surface roughening process such as that described below.

In at least some instances, and as discussed in greater detail below with reference to FIGS. 10-15, the conductive member surfaces (and/or the conductive materials used to produce the conductive members) may be magnesium sulfate (“MgSulfate”) blasted to increase the surface roughness and the effective surface area of the conductive material from its pre-processed state to achieve the desired ESA to SSA and EPESA to AW ratios.

There are a number of advantages associated with an ESA to SSA ratio that is greater than that of conventional conductive members and, for example, is at least 1.5. For example, as compared to an otherwise identical nerve cuff with conventional conductive members, adhesion by adhesives will be enhanced as a result of the higher surface roughness. The stress applied to the conductive members 120 and 126 when the nerve cuff 102 is handled or is in a furled state will be less likely to cause delamination at the relatively thin window frames 124 and 130 and/or dislodgement of the conductive members and/or bodily fluid ingress that could reach facilitates increases in the amount of stimulation energy that can be safely delivered through a window of a given size (i.e., higher current density) at a given or lower voltage and to also reduces electrode-electrolyte impedance, thereby reducing power consumption by the stimulation circuitry and increasing IPG battery life, and increased electrode capacitance. It should also be noted that reducing electrode voltage reduces the likelihood of irreversible electrode reactions, such as electrolysis, which may lead to tissue irritation and/or damage. Increasing the effective surface area of the conductive members also reduces edge effects, i.e., high current densities at the electrode edges, and the corrosion and/or tissue damage associated therewith.

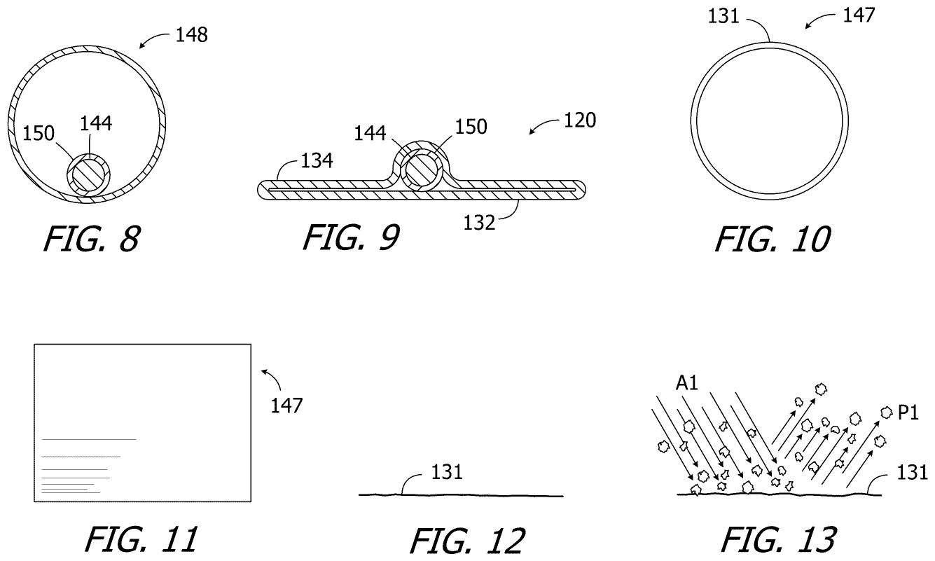

The contacts 112 and 114 in the illustrated embodiment may be individually electrically connected to the plurality contacts 108 on the lead connector 106 (FIG. 2) by wires or cables 142 (FIG. 6) that extend through the lead body 104. Each wire 142 includes a conductor 144 (FIG. 8), such as a conductor formed from MP35N material, and an insulator 146 (FIG. 6). The conductors may be connected to the rear side of the conductive members 120 and 126 by any suitable process. For example, and referring to FIGS. 8 and 9, each of the conductive members 120 (and 126) of the contacts 112 (and 114) may be formed from a tubular workpiece 148 that have been processed in the manner described below with reference to FIGS. 10-13 and that is crimped to a conductor as the tubular workpiece is compressed into a flat conductive member. Crimp tubes 150 may be provided at appropriate locations along the wires 142. The portions of the insulators 146 within the crimp tubes 150 may be removed prior to crimping (as shown) or simply squeezed out of the resulting joint during the crimping processes that form the conductive members 120 (and 126).

As is also illustrated in FIGS. 5-7, the cuff body 110 in the exemplary implementation includes a stimulation region 152 and a compression region 154. The contacts 112 and 114 are located within the stimulation region 152 and there are no contacts located within the compression region 154. The compression region 154 wraps around at least a portion of the stimulation region 152 when the nerve cuff 102 is in the pre-shaped furled state as well as in slightly larger, expanded and less lightly furled states, thereby resisting (but not preventing) expansion of the stimulation region and improving the electrical connection between the contacts 112 and 114 and the HGN. As a result, the exemplary nerve cuff 102 may be positioned around an HGN branch 24, as shown in FIG. 7, or the HGN trunk.

The exemplary cuff body 110 may be formed from any suitable material. Such materials may be biologically compatible, electrically insulative, elastic and capable of functioning in the manner described herein. The cuff materials should be pliable enough to allow a clinician to unfurl the cuff body 110 (and nerve cuff 102) and place the nerve cuff around the HGN trunk (or HGN GM branch). The exemplary materials should also be resilient enough to cause the nerve cuff body 110 (and nerve cuff 102) to return to its pre-shaped furled state when the force is removed, yet flexible enough to allow the cuff body 110 (and nerve cuff 102) to instead assume the slightly larger, expanded and less tightly furled states. By way of example, but not limitation, suitable cuff body materials include silicone, polyurethane and styrene-isobutylene-styrene (SIBS) elastomers. Suitable materials for the contacts 112 and 114 include, but are not limited to, platinum-iridium and palladium.

During one exemplary manufacturing process, the front layer 116 with the windows 122 and 128 is formed in a mold from silicone or other cuff body material. The conductive members 120 and 126, which have been previously MgSulfate blasted and connected to the wires 142, are placed into the mold over the front layer 116 with the conductive members aligned with the windows 122 and 128. The rear layer 118 is then formed in the mold from silicone or other cuff body material over the front layer 116, the conductive members 120 and 136, and portions of the wires 136. Primer may also be employed to improve adhesion and further reduce the likelihood of delamination as the nerve cuff is manipulated. For example, silicone primer may be applied to the front surfaces 132 and 136 of the conductive members 124 and 126 in the area of the window frames 124 and 130, as well to the rear surfaces 134 and 138, as is discussed in greater detail below with reference to FIGS. 16-19.

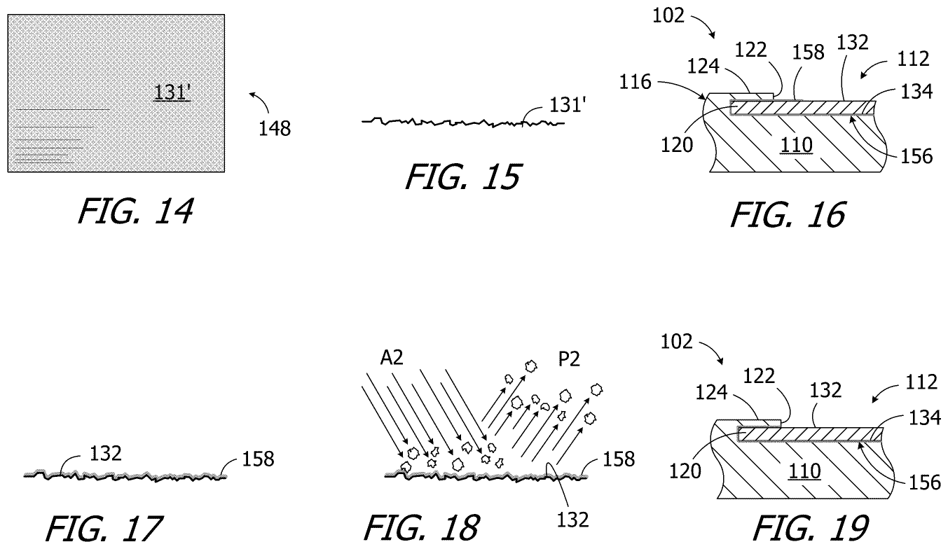

As alluded to above, the present conductive members may be formed from a tubular workpiece that is processed in such a manner that the effective surface area is increased. To that end, and referring to FIGS. 10-12, the pre-processing tubular workpiece 147 may be formed from platinum-iridium (or platinum) and has a relatively smooth, visually shiny outer surface 131 that will define the outer surfaces of the conductive member formed from the workpiece post-processing. FIG. 12 is a magnified representation of the outer surface 131. The outer surface 131 may roughened by MgSulfate blasting. Turning to FIG. 13, and as used herein, “MgSulfate blasting” is the shooting of a stream of small MgSulfate particulates P1 at a surface in a jet of air A1 and may be used to alter and clean the surface in a single step. The MgSulfate blasting transforms the pre-processing tubular workpiece 147, where the outer surface 131 has a relatively smooth, shiny state, to the post-processing tubular workpiece 148 illustrated in FIGS. 14 and 15, where the outer surface 131′ has a finely textured, visually matte state and has an ESA to SSA ratio of at least 1.5. The outer surface 131′ may be transformed to the finely textured, visually matte state in a single MgSulfate blasting process. The post-processing tubular workpiece 148 may then be compressed into a conductive member 120 (or 126) in the manner described above with reference to FIGS. 8 and 9. In other instances, the MgSulfate blasting process may occur after the relatively flat conductive member is formed and prior to assembly of the associated nerve cuff. In either instance, the conductive members 120 and 126 may be incorporated into nerve cuffs with EPESA to AW ratios that are at least 1.5 as described above.

MgSulfate blasting parameters such as air pressure, the particular hydrate of MgSulfate from which the particles are formed, particle size and size distribution, particle feed rate, particle shape, nozzle orifice diameter, air pressure, working distance and/or length of blasting time, may be selected and optimized to achieve the intended result.

Exemplary hydrates of MgSulfate that may be employed in blasting processes include, but are not limited to, the heptahydrate (MgS0₄·7H₂0), which is commonly known as Epsom salt, and the monohydrate (MgS0₄·H₂0), which is sold commercially by Crystal Mark Inc. under the tradename Burr-Blast K™. These and other hydrates of MgSulfate are advantageously both hygroscopic and water soluble, which allows any residue from a dry MgSulfate blasting process to be easily removed with a simple aqueous cleaning process, and are also biocompatible. It should also be noted that the density of Burr-Blast K™ monohydrate MgSulfate is 2.4g/cc, which is relatively high. The relatively high density is advantageous because the weight and kinetic energy of individual particles in the blast stream is greater than it is with lower density materials. This increases the aggressiveness of the blasting process. By way of example, but not limitation, particle size distributions in some implementations may range from a particle distribution with a 50 percentile value of up to 39.3 μm (i.e., 50 percent of the particles are 39.3 μm or less) to a particle distribution with a 50 percentile value of up to 111.6 um (i.e., 50 percent of the particles are 111.6 μm or less). In some instances, particles may be provided in a single quantity so that the surface being processed is struck with particles of various sizes in a single treatment, while in other instances multiple quantities with successively smaller 50 percentile values may be employed in multiple treatments.

In one specific example that may be used to transform the surface of a platinum-iridium tubular workpiece from a relatively smooth, visually shiny state to a finely textured, visually matte state that may be compressed into a conductive member with a surface having an ESA to SSA ratio of at least 1.5, the MgSulfate blasting process may be performed with a Crystal Mark MV-2 Micro Sandblaster and the following parameters: MgSulfate monohydrate, powder flow of 7 out of 10; nozzle orifice diameter of 0.032 inch; air pressure of 40 psi; working distance of 0.5 to 1.0 inch; and blasting time of about 3 seconds.

As noted above, silicone primer may be applied to the front surfaces 132 and 136 of the conductive members 120 and 126 in the area of the window frames 124 and 130, as well as to the rear surfaces 134 and 138. The present inventors have determined that some of the primer applied to the surface of the conductive members that will be aligned with the window frames 124 and 130 may enter and remain in the area that will be exposed by the windows. For example, and referring to FIGS. 16 and 17, the assembled nerve cuff 102 may include one or more contacts 112 (or 114) wherein primer 156 covers the conductive member rear surface 134 (or 138), the side surfaces, and the portion of the conductive member front surface 132 (or 136) under the window frame 124 (or 128). Additionally, a small quantity 158 of the silicone primer 156 is located on the front surface 132 (or 136) within the window 122 (or 128). Alternatively, or in addition, some of the silicone adhesive (not shown) that secures the cuff body front and rear layers 116 and 118 to one another may be present within the window 122 (or 128) on the front surface 132 (or 136). The silicone primer and/or adhesive (collectively “contaminants”) within the window frame may compromise the electrical performance and biocompatibility of the associated nerve cuff. Accordingly, an assembled nerve cuff may be subjected to a second MgSulfate blasting process that is configured to remove any contaminants that are on the front surfaces of the conductive members within the windows without damaging the window frames or other parts of the front layer.

Referring to FIGS. 18 and 19, a second MgSulfate blasting process that involves shooting a stream of small particulates P2 at the exposed primer 158 (and/or adhesive) in a jet of air A2 may be used to clean the surface 132 (or 136) within the windows 122 (or 128). The MgSulfate blasting parameters of the second MgSulfate blasting process may be different than those of the first MgSulfate basting process. For example, the MgSulfate blasting parameters may be such that the second MgSulfate blasting process removes contaminants (e.g., primer and/or adhesive flash) from the platinum-iridium contact member surfaces without damaging the window frame 124 (or 130) and other portions of the nerve cuff. In one specific example, the MgSulfate blasting process may be performed with a Crystal Mark MV-2 Micro Sandblaster and the following parameters: MgSulfate monohydrate, powder flow of 7 out of 10; nozzle orifice diameter of 0.032 inch; air pressure of 15 psi; working distance of 0.5 to1.0 inch; and blasting time of about 2 seconds.

With respect to dimensions, the exemplary nerve cuffs described herein are configured to accommodate HGN structures that have diameters of about 2.5 mm (e.g., the HGN GM branch 24), about 3.0 mm (e.g., the HGN GM branch 24 in a swollen state), and about 4.0 mm (e.g., the HGN trunk 22).

Using the nerve cuff 102 as an example, the plurality of contacts 112-1 and plurality of contacts 112-2 which, as noted above, each function as single relatively wide contact and are sized such that the relatively wide contacts will each extend completely around the inner lumen 174 (FIG. 9) defined by the nerve cuff, i.e., 360° or more around the longitudinal axis of the inner lumen, when the cuff body 110 is in the fully furled state that accommodates an HGN structure having a diameter of about 2.5 mm. Viewed as a group, the relatively narrow contacts 114 also will extend completely around the inner lumen 174 when the when the cuff body 110 is in the fully furled state. The relatively wide pluralities of contacts 112-1 and 112-2 will also extend substantially around the inner lumen 174, i.e., at least 288° in some examples and 360° or more in other examples, around the longitudinal axis of the inner lumen, when the cuff body 110 is in an expanded and less tightly furled state that accommodates an HGN structure having a diameter of about 4.0 mm, as will, when viewed as a group. the relatively narrow contacts 114.

The dimensions of the present nerve cuffs, including the various elements thereof, may be any dimensions that result in the nerve cuffs functioning as intended. With respect to the dimensions of the cuff body 110 of the exemplary nerve cuff 102, and referring to FIG. 5, the cuff body is about 1.1 inches wide and about 0.34 inches long. The width of the stimulation region 148 is about 0.6 inches, while the width of the compression region 150 is about 0.4 inches. The individual contacts 112 are same size, and the relatively narrow contacts 114 are the same size, in the illustrated implementation. In other implementations, the contacts 112 may be different sizes and/or the relatively narrow contacts 114 may be different sizes.

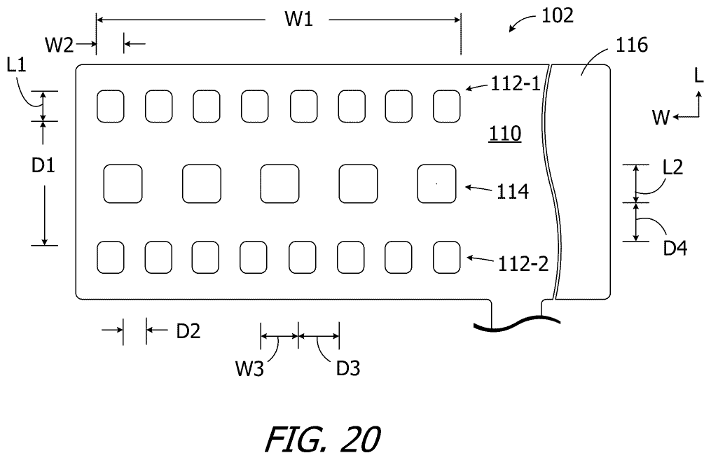

Referring to FIG. 20, the width W1 of the total width each group of contacts 112-1 and 112-2 is about 0.5 inches, the length L1 is about 0.04 inches, the width W2 is about 0.03 inches, the effective surface area is at least 0.0024 square inches, the distance D1 between contacts 112-1 and contacts 112-2 is about 0.2 inches, and the distance D2 between adjacent contacts 112 is about 0.02 inches. The width W3 of the contacts 114 is about 0.07 inches, the length L2 is about 0.07 inches, the effective surface area is at least 0.001 square inches, and the distance D3 between the contacts 114 is about 0.05 inches. The distance D3 may also be increased or decreased as desired to accomplish various stimulation objectives. The distance D4 between the contacts 114 and the contacts 112-1 and 112-2 is about 0.06 inches. It should be noted that the present contacts, conductive members, cuff bodies and nerve cuffs are not limited to the exemplary embodiments described above. By way of example, the sizes, shapes and spacings of the conducive members and the windows (and, therefore, the contacts) may be varied.

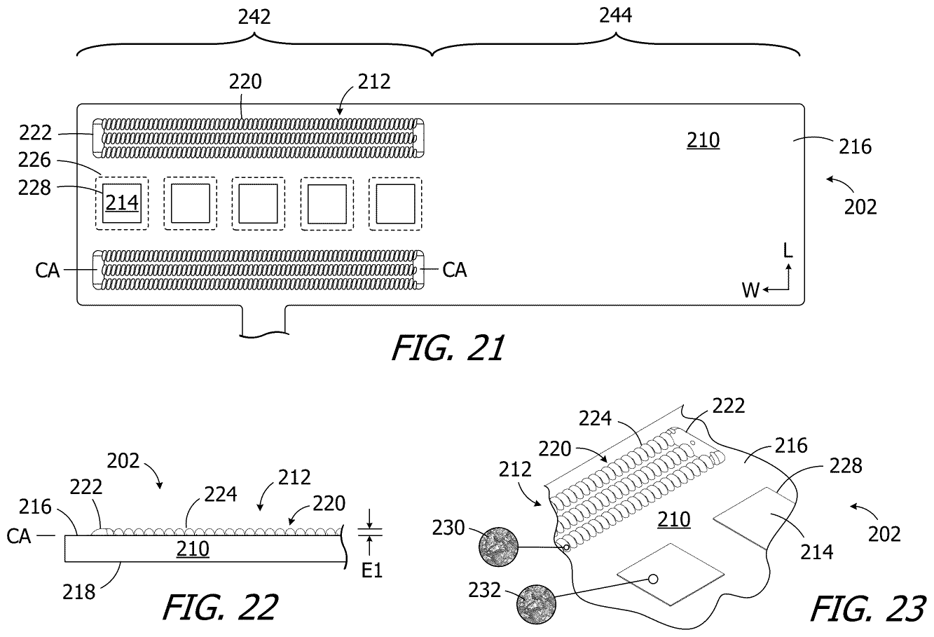

Another exemplary nerve cuff is generally represented by reference numeral 202 in FIGS. 21-24. The exemplary nerve cuff 202 is similar to nerve cuff 102 and similar elements are represented by similar reference numerals. The nerve cuff 202 also has furled state similar to that illustrated in FIG. 7.

The exemplary nerve cuff 202 includes a cuff body 210 that defines a length L and a width W that is greater than the length, first and second relatively wide electrically conductive coil contacts (or “relatively wide coil contacts” or “coil contacts”) 212 on the cuff body 210 that extend in the width direction and are spaced from one another in the length direction and a plurality of relatively narrow electrically conductive flat contacts (or “relatively narrow contacts” or “contacts”) 214. Such coil contacts and flat contacts may also be referred to as “electrodes.” Although the number may increase or decrease in the context of other nerve applications, at least five relatively narrow contacts 214 may be spaced from one another in the width direction are located between the first and second relatively wide coil contacts 212, and there are five relatively narrow contacts 214 in the illustrated embodiment. In the implementation illustrated in FIGS. 21-24, the relatively narrow contacts 214 are centered relative to the relatively wide coil contacts 212 and are aligned with one another in the length direction. In other implementations, the relatively narrow contacts may be non-centered relative to the relatively wide coil contacts 212 and/or offset from one another in the length direction. With respect to shape, and although the present inventions are not so limited, the relatively wide coil contacts 212 have an overall rectangular shape, while the relatively narrow contacts 214 are squares.

The exemplary cuff body 210 includes a front layer 216 that will face the HGN trunk or branch and a rear layer 218 that will face away from the HGN trunk or branch. The outer surface of the front layer 216, i.e. the top surface in FIG. 22, defines the front surface of the cuff body. The outer surface of the rear layer 218, i.e. the bottom surface in FIG. 22, defines the rear surface of the cuff body. The exemplary coil contacts 212 each include a plurality of coil-shaped electrically conductive members (or “coil-shaped conductive members” or “conductive members”) 220 that are electrically connected to one another in parallel by welds 222 or any other suitable structure. The welds 222 may be located at the longitudinal ends of the coil-shaped conductive members 220, as shown, or in another location. Other exemplary coil contacts may include a one, two or more than three coil-shaped conductive members 220. The coil-shaped conductive members 220 may be helical (as shown) or otherwise spiral and define a central axis CA. Although other shapes may be employed, the central axes CA of the coil-shaped conductive members 220 are straight lines and the coil contacts 212 are linear. The coil-shaped conductive members 220 may be oriented in such a manner that the central axes CA are parallel to one another (as shown) or non-parallel. The central axes CA are straight in the implementation illustrated in FIGS. 21-24, but may be curved or have both straight and curved portions in other implementations. The coil-shaped conductive members 220 may be positioned on the cuff body 210 in such a manner that the central axes CA lie in a common plane such as, for example, the plane defined by the outer surface of the front layer 216 (as shown) or may lie in different planes that are defined by the length L and width W. The coil-shaped conductive members 220 project outwardly from the cuff body 210 by a distance E1 (FIG. 22) and, accordingly, have exposed portions 224 that project outwardly from the cuff body 210 and are not covered by cuff body material or other electrically insulating material. Put another way, the coil-shaped conductive members 220 are only partially embedded in the cuff body 210, such there are embedded portions and non-embedded portions, and the exposed, non-embedded portions of the coil-shaped conductive members together define the coil contacts 212. The extent of the exposure of the exposed portions 224 may be varied in order to vary the levels of tissue interaction with the coil contacts 212.

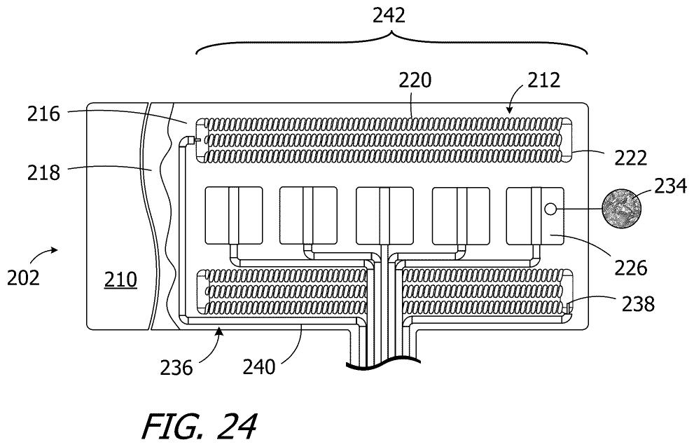

With respect to the contacts 214, the exemplary nerve cuff 202 includes five relatively narrow conductive members 226 that are located between the front layer 216 and rear layer 218. Portions of the relatively narrow conductive members 226 are exposed by way of respective relatively narrow openings 228 in the cuff body front layer 216, thereby defining the contacts 214. The openings 228 extend from the outer surface of the front layer 216 to the associated conductive members 226. The conductive members 226 may be formed in the manner described above with reference to FIGS. 8 and 9 from tubular workpieces that have been processed in the manner described above with reference to FIGS. 10-13 and that are crimped to a conductor 238 (FIG. 24) as the tubular workpiece is compressed into a flat conductive member.

Referring to FIGS. 22 and 23, the exposed portions 224 of the coil-shaped conductive members 220 have front surfaces 230 that have been altered, in the manner described below, to increase the effective surface area (“ESA”) in the manner described below. Each front surface 230 also defines a smooth surface area (“SSA”) that is the area of a perfectly smooth surface having the size and shape of the front surface 230. The front surfaces 230 of the exposed portions 224 of the coil-shaped conductive members 220 and the front and rear surfaces 232 and 234 of the flat conductive members 226 define respective ESA to SSA ratios that are greater than that of conventional conductive members and are at least 2.0 in the exemplary implementation. The ESA to SSA ratios in other implementations may be at least 1.5, or at least 3.0, or at least 4.0, or at least 5.0, or at least 6.0, or at least 7.0, or at least 8.0, or at least 9.0, or at least 10.0. Additionally, the portions of the front surfaces 232 that are exposed by the windows 228 have an exposed portion effective surface area (“EPESA”) to area defined by the perimeters of the windows (“AW”) ratio is greater than that of conventional conductive member and window combinations and is at least 1.5 in the exemplary implementations. The EPESA to AW ratio in other implementations may be at least 2.0, or at least 3.0, or at least 4.0, or at least 5.0, or at least 6.0, or at least 7.0, or at least 8.0, or at least 9.0, or at least 10.0.

Referring more specifically to FIG. 24, the coil contacts 212 and contacts 214 in the exemplary nerve cuff 202 may be individually electrically connected to a plurality contacts on a lead connector (FIG. 2) by wires 236 that extend through the associated lead body. Each wire 236 includes a conductor 238 and an insulator 240. The conductors 238 may be connected to the rear side of the welds 222 (or to the coil-shaped conductive members 220) through the use of welding or other suitable processes. The conductive members 226 may be crimped to the conductors 238 within the individual wires 236 through the use of crimp tubes in the manner described above with reference to FIGS. 8 and 9. In other implementations, the coil contacts 212 may also be electrically connected to one another by a short wire. Here, only one of the coil contacts 212 will be connected to a contact on a lead connector by way of a wire 236. In other implementations, cables may be employed in place of the wires 236.

The cuff body 210 in the exemplary implementation illustrated in FIGS. 21-24 includes a stimulation region 242 and a compression region 244, and the coil contacts 212 and contacts 214 are located within the stimulation region 242. There are no contacts located within the compression region 244. The compression region 244 wraps around at least a portion of the stimulation region 242 in the manner described above with reference to FIGS. 5-7, thereby improving the electrical connection between the coil contacts 212 and contacts 214 and the HGN.

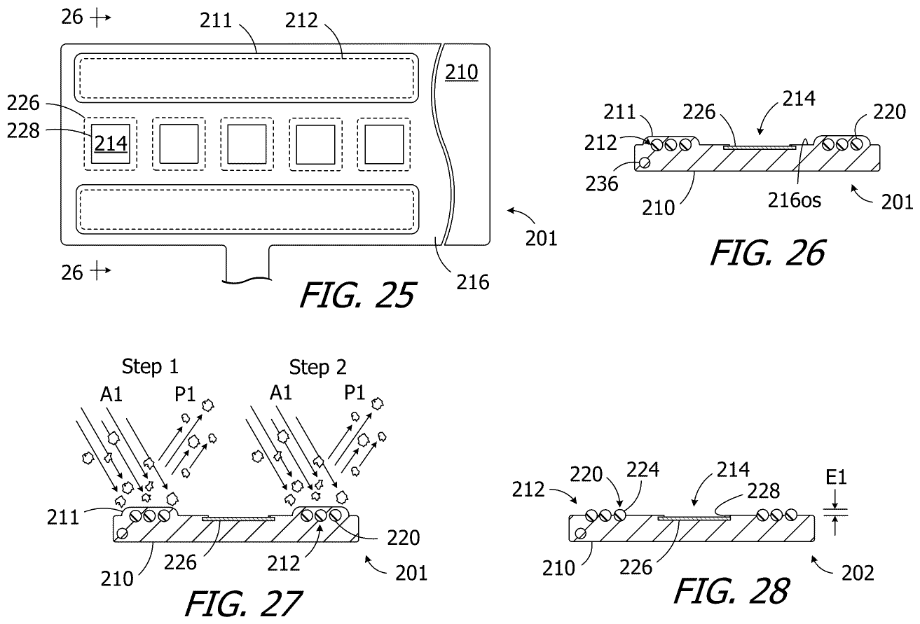

Turning to FIGS. 25 and 26, the exemplary nerve cuff 202 may be manufactured through a process that employs a cuff blank such as the cuff blank 201. The cuff blank 201 includes the various components of the nerve cuff 202, i.e., cuff body 210, coil-shaped conductive members 220, the conductive members 226, and the wires 236. The coil-shaped conductive members 220 are embedded within the cuff blank 201. In particular, the cuff body 210 of the cuff blank 201 includes caps 211 that cover the portions of the coil-shaped conductive members 220 that extend beyond the front layer outer surface 216os. The cap 211 is formed when the coil-shaped conductive members 220 are molded into the corresponding portions of the cuff body 210 and, accordingly, may be formed from the same material (e.g., silicone) as the remainder of the cuff body. For example, the coil-shaped conductive members 220 may be positioned within and bonded to grooves formed in the cuff body 210, or the coil-shaped conductive members may be pressed into uncured cuff body material. In some instances, a primer may be applied to the coil-shaped conductive members 220 prior to molding to enhance the adhesion of the cuff body material to the coils 220. Some or all of the cap 211 may be removed to expose the portions 224 of the coil-shaped conductive members 220 that define the contacts 212 as is described below with reference to FIGS. 27 and 28. Alternatively, the coil-shaped conductive members 220 may be located entirely below the front layer outer surface 216os. Here, portions of the cuff blank 201 (or the cuff blank 201a in FIG. 32) below the front layer outer surface 216os will be removed to expose portions 224 of the coil-shaped conductive members 220.

In one exemplary implementation, the exemplary coil-shaped conductive members 220 may be formed from a wire that is about 0.002 inch in diameter that is wound into a 0.50 inch long helical coil with outer diameter of about 0.009 inch and a coil pitch of about 0.003 inch. The coil-shaped conductive members 220 are positioned within the blank 201 in such a manner that ½ of the coil is above the front layer outer surface 216os, and ½ of the coil is below the front layer outer surface 216os. As such, up to 0.01 inch of the circumference of the coil-shaped conductive members 220 may be exposed in the exemplary implementation.

A MgSulfate blasting process may be employed to both expose the portions of the coil-shaped conductive members 220 and to roughen and clean the surfaces of the exposed portions 224 of the coil-shaped conductive members 220, which have a relatively smooth, visually shiny outer surface prior to the blasting process, thereby increasing the effective surface area of the exposed portions 224. For example, and referring to FIG. 27, a stream of small MgSulfate particulates P1 may be shot at the caps 211 in a jet of air A1. This may occur in a two-step process where some or all of one of the caps 211 is removed in Step 1 and then some or all of the other cap 211 is removed in Step 2. A CNC system capable of tightly controlling the position and speed of the grit blasting head may be employed. The completed nerve cuff 202 is shown in FIG. 28.

The exemplary MgSulfate blasting process illustrated in FIG. 27 may employ the same MgSulfate blasting parameter values described above with reference to FIGS. 10-15, with the exception of blasting time. The blasting time here will be greater because time is required to remove some or all of the cap 211, which may be formed from silicone or another soft material that can absorb some of the kinetic energy associated with the particles. A softer silicone (e.g., 10A-30A) will take longer to be abraded by the MgSulfate particles than a harder silicone (e.g., 60A-80A). It should also be noted that the surface texturizing (or “roughing”) and cleaning of the coil-shaped conductive members 220 will occur relatively quickly once the cap material is removed. As such, care should be taken to limit the abrading of the coil-shaped conductive members 220 to that necessary to create the desired roughness and to prevent abrading all the way through the coil-shaped conductive members.

With respect to dimensions, the dimensions of various elements of the nerve cuff 202 may be similar to or the same as the dimensions of the corresponding elements of the nerve cuff 102.

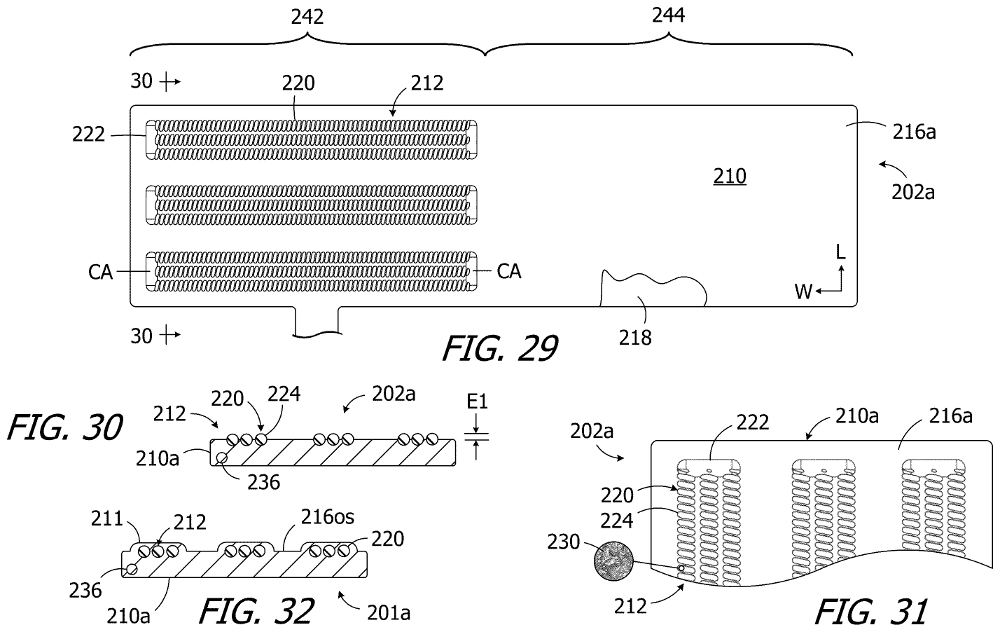

Another exemplary nerve cuff is generally represented by reference numeral 202a in FIGS. 29-31. The exemplary nerve cuff 202a is similar to nerve cuff 202 and similar elements are represented by similar reference numerals. Here, however, the flat contacts 214 replaced by an additional coil contact 212.

To that end, the exemplary nerve cuff 202a, which has furled state similar to that illustrated in FIG. 7, includes a cuff body 210a that defines a length L and a width W that is greater than the length, as well as first, second and third relatively wide coil contacts 212 on the cuff body 210a that extend in the width direction and are spaced from one another in the length direction. The exemplary cuff body 210a includes a front layer 216a that will face the HGN trunk or branch and a rear layer 218 that will face away from the HGN trunk or branch. The cuff body 210a also includes stimulation and compression regions 242 and 244.

The exemplary coil contacts 212, which include coil-shaped conductive members 220, welds 222 and exposed portions 224, are described above with reference to FIGS. 21-24. The coil contacts 212 may be individually electrically connected to a plurality contacts on a lead connector (FIG. 2) by wires 236 in the manner described above with reference to FIG. 24. The exposed portions 224 have front surfaces 230 that have been altered, in the manner described above, to increase their ESA to SSA ratios that are greater than that of conventional conductive members and are at least 2.0 in the exemplary implementation. The ESA to SSA ratios in other implementations may be at least 1.5, or at least 3.0, or at least 4.0, or at least 5.0, or at least 6.0, or at least 7.0, or at least 8.0, or at least 9.0, or at least 10.0. 0.

The exemplary nerve cuff 202a may be manufactured through a process that employs a cuff blank, such as the cuff blank 201a illustrated in FIG. 32. The cuff blank 201a includes the various components of the nerve cuff 202a, i.e., the cuff body 210a, the coil-shaped conductive members 220 are embedded under the caps 211 (or under the front layer outer surface 216os in those instances where the coil-shaped conductive members 220 will be located entirely below the front layer outer surface 216os), and the wires 236. As described above with reference to FIGS. 27 and 28, a MgSulfate blasting process may be employed to both expose the portions of the coil-shaped conductive members 220 and to roughen and clean the surfaces of the exposed portions 224 of the coil-shaped conductive members 220, which have a relatively smooth, visually shiny outer surface prior to the blasting process, thereby creating the roughed front surfaces 230 and increasing the effective surface area of the exposed portions 224.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions. The inventions include any and all combinations of the elements from the various embodiments disclosed in the specification. The scope of the present inventions is limited solely by the claims set forth below.

1-21. (canceled)

22. A method, comprising:
MgSulfate blasting a portion of a nerve cuff blank, that includes a cuff body and at least one coil-shaped conductive member embedded in the cuff body, in such a manner that a portion of the coil-shaped conductive member is exposed.

23. A method as claimed in claim 22, wherein
the exposed portion of the coil-shaped conductive member has a front surface that defines a smooth surface area (“SSA”) and an effective surface area (“ESA”); and
the front surface of the exposed portion of the coil-shaped conductive member defines an ESA to SSA ratio that is at least 1.5.

24. A method as claimed in claim 22, wherein
the nerve cuff blank includes a plurality of coil-shaped conductive members; and
the portion of the nerve cuff is MgSulfate blasted in such a manner that a portion of each of the plurality of coil-shaped conductive members is exposed.

25. A method as claimed in claim 24, wherein
the exposed portions of the coil-shaped conductive members each have a front surface that defines a smooth surface area (“SSA”) and an effective surface area (“ESA”); and
the front surfaces of the exposed portions of the coil-shaped conductive members define an ESA to SSA ratio that is at least 1.5.

26. A method as claimed in claim 23, wherein
the ESA to SSA ratio is selected from the group consisting of at least 1.5, at least 2.0, at least 3.0, at least 4.0, at least 5.0, at least 6.0, at least 7.0, at least 8.0, at least 9.0, and at least 10.0.

27. A method as claimed in claim 24, wherein
the cuff body defines a length, a length direction, a width that is greater than the length, and a width direction;
the plurality of coil-shaped conductive members comprises at least a first coil-shaped conductive member that extends in the width direction and at least a second coil-shaped conductive member that extends in the width direction and is separated from the first coil-shaped conductive member in the length direction; and
the nerve cuff blank includes a plurality of conductive members that are located between the first and second coil-shaped conductive members and are spaced from one another in the width direction.

28. A method as claimed in claim 27, wherein
the plurality of conductive members that are located between the first and second coil-shaped conductive members comprise flat conductive members.

29. A method as claimed in claim 27, wherein
the plurality of conductive members that are located between the first and second coil-shaped conductive members include MgSulfate blasted front surfaces.

30. A method as claimed in claim 27, wherein
the plurality of conductive members that are located between the first and second coil-shaped conductive members include MgSulfate blasted rear surfaces.

31. A method as claimed in claim 27, wherein
the at least a first coil-shaped conductive member comprises a plurality of first coil-shaped conductive members that are electrically connected to one another; and
the at least a second coil-shaped conductive member comprises a plurality of second coil-shaped conductive members that are electrically connected to one another.

32. A method as claimed in claim 24, wherein
the cuff body defines a length, a length direction, a width that is greater than the length, and a width direction;
the plurality of coil-shaped conductive members comprises a first electrically conductive coil contact that extends in the width direction and includes at least two coil-shaped conductive members and second electrically conductive coil contact that extends in the width direction, includes at least two coil-shaped conductive members and is separated from the first electrically conductive coil contact in the length direction.

33. A method as claimed in claim 32, wherein
the plurality of coil-shaped conductive members further comprises a third electrically conductive coil contact that extends in the width direction, includes at least two coil-shaped conductive members and is located between the first and second electrically conductive coil contacts.
